(19) United States
(12) Patent Application Publication
(10) Pub. No.: US 2019/0282677 A1
(43) Pub. Date: Sep. 19, 2019

(54) METHOD OF PREVENTING OR REDUCING BIOFILM FORMATION

(71) Applicants: Lokender KUMAR, Golden, CO (US); Susanta K. SARKAR, Highlands Ranch, CO (US)

(72) Inventors: Lokender KUMAR, Golden, CO (US); Susanta K. SARKAR, Highlands Ranch, CO (US)

(21) Appl. No.: 16/355,090

(22) Filed: Mar. 15, 2019

Related U.S. Application Data
(60) Provisional application No. 62/643,614, filed on Mar. 15, 2018.

(51) Int. Cl. A61K 38/48; A61K 45/06; A01N 37/18

(52) U.S. Cl. CPC A61K 38/4886; C12Y 304/24024; A01N 37/18; A61K 45/06

(57) ABSTRACT

A method of disrupting or reducing a biofilm, the methods comprising contacting the biofilm with a matrix metalloproteinase (MMP). The biofilm may be present on inanimate or biological surfaces. Also, the MMP may be used to prevent the growth of a biofilm on an item, such as a medical device, by applying the MMP to the item. Also, therapeutic compositions comprising the MMP, which may be used to treat an individual having a biofilm-associated disease or disorder.

### CROSS REFERENCE TO RELATED APPLICATION

This application claims priority and the benefit under 35 U.S.C. § 119(e) to U.S. Provisional Patent Application Ser. No. 62/643,614 filed Mar. 15, 2018, which is incorporated herein in its entirety by reference.

### TECHNICAL FIELD

The present invention relates to a novel method for disrupting or reducing a biofilm, as well as treating an individual having a biofilm-associated disease or disorder. The present invention also related to medical devices treated to prevent biofilm formation, kits containing the medical device, and methods of preparing the same.

### BACKGROUND

The Centers for Disease Control and Prevention (CDC) estimates that there are at least 2 million antibiotic-resistant infections annually in the U.S. resulting in around 23,000 deaths. Bacteria develop resistance to antimicrobial agents by evolving molecular mechanisms including targeted mutations, efflux pumps, and enzyme modifications. Bacteria that are not innately resistant to antibiotics may also become resistant by forming persistent biofilms that lead to chronic infections. The National Institute of Health reports that 80% of total human bacterial infections are biofilm-associated. Biofilms are surface-associated, three dimensional bacterial communities surrounded by an extracellular matrix that protect cells from antibiotics and immune cell attack. Biofilm matrices act as physical barriers to antibiotics and create a favorable ecological niche for long-term survival under harsh environmental and nutrient-poor conditions. As such, biofilm-associated infections can become highly resistant to antibiotic therapy. Thus, there is a need for improved methods of treating such infections.

Human matrix metalloproteases (MMPs) are essential for tissue remodeling and can degrade a wide range of matrix and non-matrix associated proteins. In particular, MMP1, a collagenase that is known to degrade type-1 collagen, can also degrade various structural components of the extracellular matrix (ECM). MMP1 has also been shown to play a role in the immune response to HIV, Hepatitis B, Helicobacter pylori, and Mycobacterium tuberculosis and in inflammation. Thus, the present disclosure provides methods of using MMPs to disrupt biofilms, and to treat individuals having a biofilm-associated disease or disorder.

### SUMMARY

The present disclosure provides a method of disrupting or reducing a biofilm, the method comprising contacting the biofilm with a matrix metalloproteinase protein (MMP). The MMP may be any MMP capable of degrading at least one component in the biofilm. The MMP may be from a plant, an invertebrate, or a mammal. The MMP may be a gelatinase, a collagenase, a stromelysin, or a film-sort MMP. The MMP may be MMP1, MMP2, MMP3, MMP7, MMP8, MMP9, MMP10, MMP11, MMP12, MMP13, MMP14, MMP15, MMP16, MMP17, MMP18, MMp19, MMP20, MMP21, MMP23A, MMP23B, MMP24, MMP25, MMP26, MMP27, or MMP28.

The biofilm may comprise any microorganism capable of forming a biofilm. The microorganism may be a bacteria or a fungus. The bacteria may be a Gram-negative bacteria or a Gram-positive bacteria. The Bacteria may come from a genus selected from the group consisting of Enterococcus, Streptococcus, Staphylococcus, Bacillus, Lactobacillus, Lactococcus, Escherichia, Pseudomonas, Haemophilus, Klebsiella, Porphyromonas, and combinations thereof. The bacteria may be Enterococcus faecalis, Escherichia coli, Pseudomonas aeruginosa, Streptococcus pneumonia, Streptococcus viridans, Staphylococcus aureus, Staphylococcus epidermidis, Klebsiella pneumoniae, Haemophilus influenza, Listeria monocytogenes, Lactobacillus plantarum, Lactococcus lactis, Porphyromonas gingivali, and combinations thereof.

The biofilm may be present on an inanimate surface or a biological surface. The inanimate surface may be a table, a door, a wall, a counter, a handle, a pad, an instrument or tool (e.g., a surgical instrument or tool), or a medical device. Examples of medical devices include, but are not limited to, an ultrasound machine, a hemodialysis machine, a contact lens, suture material, a probe, a catheter, a joint prostheses, a bone implant, a dental implant, surgical mesh, a surgical screw or rod, a shunt (e.g., a ventricular shunt), a heart valve, a pacemaker, a defibrillator, or a breast implant.

The biofilm may be present on a biological surface, which may be any internal or external part of a body. The biofilm may be present in, or on, the bladder, urinary tract, bone, including the medullary cavity, skin, heart tissue, including heart valves, eye tissue, sinus tissue, vascular tissue, airway tissue, lung tissue, mucous membranes, oral tissue, teeth, and sinus tissue. The biofilm may be present in a wound, such as, incisions, lacerations, avulsions, abrasions, punctures, and burns.

The step of contacting may comprise contacting a therapeutic composition comprising the MMP with the biofilm. The therapeutic composition may comprise sterile liquids, such as, water, oil, polyethylene glycol (PEG), dextrose, glycerol, buffers, stabilizers, detergents, dispersants, surfactants, antimicrobial agents, enzymes other than MMPs, biocides, or chelating agents. The therapeutic composition may be formulated in a liquid form, or as a paste, a slurry, a gel, an emulsion, a lotion, an emollient, a spray, or a rinse. The therapeutic composition may comprise more than one MMP. The therapeutic composition may comprise an amount of MMP sufficient to alleviate one symptom of a disease or disorder resulting from the biofilm.

The present disclosure also provides a method of treating an individual for a biofilm-associated disease or disorder, the method comprising administering the MMP to the individual such that the MMP makes contact with the biofilm. The method may comprise administering to the individual a therapeutic composition comprising an amount of MMP sufficient to alleviate one symptom of a disease or disorder resulting from the biofilm (i.e., therapeutically effective amount). The therapeutic composition may comprise more than one MMP.

The present disclosure also provides a therapeutic composition comprising at least one MMP, for practicing the disclosed methods. The therapeutic composition may comprise sterile liquids, such as, water, oil, polyethylene glycol (PEG), dextrose, glycerol, buffers, stabilizers, detergents, dispersants, surfactants, antimicrobial agents, enzymes other than MMPs, biocides, or chelating agents. The therapeutic composition may be formulated in a liquid form, or as a paste, a slurry, a gel, an emulsion, a lotion, an emollient, a spray, or a rinse. The therapeutic composition may comprise more than one MMP. The therapeutic composition may comprise an amount of MMP sufficient to alleviate one symptom of a disease or disorder resulting from the biofilm.

The present disclosure also provides a method for preventing biofilm formation, the method comprising applying to the surface of an item an amount of MMP effective to prevent the formation of a biofilm. The item may be a medical device, such as an ultrasound machine, a hemodialysis machine, a contact lens, suture material, a probe, a catheter, a joint prostheses, a bone implant, a dental implant, surgical mesh, a surgical screw or rod, a shunt (e.g., a ventricular shunt), a heart valve, a pacemaker, a defibrillator, or a breast implant. More than one MMP may be applied to the item. The effective amount of MMP may be applied as a composition that may be a liquid, a buffered solution, a paste, a slurry, a gel, a lotion, an emollient, a spray, a rinse, or an emulsion. More than one MMP may be applied to the surface to of the item.

The present disclosure also a method of inhibiting the growth of a microorganism, the method comprising contacting the organism with a matrix metalloproteinase (MMP). The microorganism may be present on an inanimate surface or a biological surface, or it may be present in a solution. The microorganism may be a bacteria or a fungus. The bacteria may be a Gram-negative bacteria or a Gram-positive bacteria. The Bacteria may come from a genus selected from the group consisting of Enterococcus, Streptococcus, Staphylococcus, Bacillus, Lactobacillus, Lactococcus, Escherichia, Pseudomonas, Haemophilus, Klebsiella, Porphyromonas, and combinations thereof. The bacteria may be Enterococcus faecalis, Escherichia coli, Pseudomonas aeruginosa, Streptococcus pneumonia, Streptococcus viridans, Staphylococcus aureus, Staphylococcus epidermidis, Klebsiella pneumoniae, Haemophilus influenza, Listeria monocytogenes, Lactobacillus plantarum, Lactococcus lactis, Porphyromonas gingivali, and combinations thereof. The MMP may be contacted with the microorganism in the form of a composition such as a therapeutic composition of the disclosure.

The present disclosure also provides an item, such as a medical device, tubing, a cannula, a probe, a catheter, a joint prostheses, a bone implant, a dental implant, surgical mesh, a surgical screw or rod, a shunt (e.g., a ventricular shunt), a heart valve, a pacemaker, a defibrillator, or a breast implant, comprising on at least one surface, an amount of MMP sufficient to prevent the formation of a biofilm. The item may be wound dressing, such as gauze, a hydrocolloid dressing, a hydrogel dressing, or an alginate dressing. The MMP may be any MMP of the disclosure that is capable of degrading at least one component in the biofilm.

The present disclosure also provides a kit for practicing methods of the disclosure, wherein the kit comprises at least one MMP intended for use in disrupting or reducing a biofilm, preventing the formation of a biofilm on an item, treating an individual for a biofilm-associated disease or disorder, or inhibiting the growth of a microorganism. The kit may comprise instructions for using the MMP to disrupt or reduce a biofilm, prevent the formation of a biofilm on an item, inhibit the growth of a microorganism, treat an individual for a biofilm-associated disease or disorder, or inhibit the growth of a microorganism. The kit may comprise any known MMP, as long as it is capable of degrading at least one component in the biofilm. The MMP may be from a plant, an invertebrate or a vertebrate. The MMP may be from a mammal, such as a human. The MMP may be a gelatinase, such as MMP2 or MMP9. The MMP may be a collagenase, such as MMP1, MMP8, MMP13 or MMP18. The MMP may be a stromelysin. The MMP may be selected from the group consisting of MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. The MMP may be in a liquid solution, a buffered solution, a paste, a slurry, a gel, a lotion, an emollient, a spray, a rinse, or an emulsion. The kit may comprise a therapeutic composition for treating an individual for a biofilm-associated disease or disorder. The kit may comprise more than one MMP. In addition, the kit may comprise tubes, syringes, needles, vials, diluents, and the like, for practicing methods of the disclosure.

### BRIEF DESCRIPTION OF FIGURES

FIGS. 1A-G. Inhibitory effect of MMP1 on E. faecalis biofilms. (A-G) Absorption of CV stain at 635 nm (OD₆₃₅) of MMP1-treated biofilms of E. faecalis FA2-2 and V583 strains over seven days. Experiments with BHI media and protein buffer were used as controls. For inhibition experiments, biofilms were grown in presence of MMP1 day 1 (FIG. 1A), day 2 (FIG. 1B), day 3 (FIG. 1C), day 4 (FIG. 1D), day 5 (FIG. 1E), day 6 (FIG. 1F), and day 7 (FIG. 1G). Typical images of wells in microtiter plates are given at the top of each panel. * indicates p-value: *<0.01, **<0.01, and ***<0.001. Error bars on the data points represent the standard deviations of 3 technical repeats.

FIGS. 2A-G. Disruptive effect of MMP1 on E. faecalis biofilms. (A-G) Absorption of CV stain at 635 nm (OD₆₃₅) of MMP1-treated biofilms of FA2-2 and V583 strains over seven days—day 1 (FIG. 2A), day 2 (FIG. 2B), day 3 (FIG. 2C), day 4 (FIG. 2D), day 5 (FIG. 2E), day 6 (FIG. 2F), and day 7 (FIG. 2G). Experiments with BHI media and protein buffer were used as controls. For disruption experiments, biofilms were first grown in BHI media without MMP1 for desired duration, followed by MMP1 treatment. Typical images of wells in microtiter plates are given at the top of each panel. * indicates p-value: *<0.01, **<0.01, and ***<0.001; ns indicates that the effect is not significant. Error bars on the data points represent the standard deviations of 3 technical repeats.

FIGS. 3A & 3B. SEM micrographs of established E. faecalis biofilms. Biofilms were first grown for 3 to 7 days and then treated with MMP1. FIG. 3A illustrates vancomycin susceptible strain FA2-2 in BHI media, protein buffer or MMP1. FIG. 3B illustrates vancomycin resistant strain V583 in BHI media, protein buffer or MMP1. In comparison to the control experiments, active MMP1 led to disruption of biofilms resulting in more empty spaces without any bacteria.

FIGS. 4A-D. Colony forming unit (CFU) assay to quantify viable cells in E. faecalis biofilms. (A-D) Quantification (Log ₁₀ CFU) of live bacterial cells in 1, 3, 5 and 7 day old E. faecalis FA2-2 and V583 biofilms treated with and without MMP1 under inhibition conditions. Biofilms were grown in presence of MMP1 from day 0 to day 7—day 1 (FIG. 4A), day 3 (FIG. 4B), day 5 (FIG. 4C), and day 7 (FIG. 4D). * indicates p-value: *p<0.01, **p<0.01 and ***p<0.001; ns indicates that the effect is not significant. Error bars on the data points represent the standard deviations of 3 technical repeats.

FIGS. 5A-D. Colony forming unit (CFU) assay to quantify viable cells in E. faecalis biofilms. (A-D) Quantification (Log ₁₀ CFU) of live bacterial cells in 1 day (FIG. 5A), 3 day (FIG. 5B), 5 day (FIG. 5C) and 7 day (FIG. 5D) E. faecalis FA2-2 and V583 biofilms treated with and without MMP1 under disruption conditions. Biofilms were first grown in BHI media without MMP1 for desired duration, followed by MMP1 treatment. * indicates p-value: *p<0.01, **p<0.01 and ***p<0.001; ns indicates that the effect is not significant. Error bars on the data points represent the standard deviations of 3 technical repeats.

FIGS. 6A-D. Congo red staining assay to quantify effects of MMP1 on E. faecalis biofilms. (A-D) Absorption of Congo red stain at 490 nm (OD₄₉₀) of 1 day (FIG. 6A), 3 day (FIG. 6B), 5 day (FIG. 6C) and 7 day (FIG. 6D)E. faecalis FA2-2 and V583 biofilms treated with and without MMP1 under inhibition conditions Experiments with BHI media and protein buffer were used as controls. Biofilms were grown in presence of MMP1 from day 0 to day 7 (1 day—FIG. 6A, 3 day—FIG. 6B, 5 day—FIG. 6C, and 7 day—FIG. 6D). Typical images of wells in microtiter plates are given at the top of each panel. * indicates p-value: *p<0.01, **p<0.01 and ***p<0.001; ns indicates that the effect is not significant. Error bars on the data points represent the standard deviations of 3 technical repeats.

FIGS. 7A-D. Congo red staining assay to quantify effects of MMP1 on E. faecalis biofilms. (A-D) Absorption of Congo red stain at 490 nm (OD₄₉₀) of 1 day (FIG. 7A), 3 day (FIG. 7B), 5 day (FIG. 7C) and 7 day (FIG. 7D)E. faecalis FA2-2 and V583 biofilms treated with and without MMP1 under disruption conditions Experiments with BHI media and protein buffer were used as controls. For inhibition experiments, biofilms were first grown in BHI media without MMP1 for desired duration, followed by MMP1 treatment. Typical images of wells in microtiter plates are given at the top of each panel. * indicates p-value: *p<0.01, **p<0.01 and ***p<0.001; ns indicates that the effect is not significant. Error bars on the data points represent the standard deviations of 3 technical repeats.

FIGS. 8A & B. Effect of MMP1 on E. faecalis growth. Growth curves of E. faecalis in presence of active MMP1, BHI media, and protein buffer for (A) FA2-2 and (B) V583 strains respectively. Symbols represent data points. Solid lines are fits to the logistic equation of bacterial growth, y=a/{1+b exp(−kt)}, where k represents the growth rate. Error bars on the data points represent the standard deviations of 6 technical repeats. For FA2-2 strain, the best fit parameters are: a=0.85±0.01, b=24.98±4.44, k=1.38±0.08 (BHI media); a=0.81±0.01, b=32.53±7.02, k=1.49±0.09 (protein buffer); a=0.80±0.01, b=36.62±8.88, k=1.53±0.10 (inactive MMP1); a=0.71±0.02, b=8.47±1.22, k=0.79±0.06 (active MMP1). For V583 strain, the best fit parameters are: a=0.96±0.01, b=13.41±1.20, k=1.05±0.04 (BHI media); a=0.92±0.01, b=7.70±0.62, k=0.85±0.04 (protein buffer); a=0.92±0.01, b=7.73±0.65, k=0.88±0.04 (inactive MMP1); a=0.75±0.01, b=10.12±1.21, k=0.96±0.05 (MMP1). For fit parameters, error bars represent the standard error of the mean.

FIG. 9. SEM micrographs of established E. faecalis biofilms. Biofilms of vancomycin susceptible strain FA2-2 grown for 3-7 days and then treated with MMP1. The media was BHI media, protein buffer or MMP1.

FIG. 10. SEM micrographs of established E. faecalis biofilms. Biofilms of vancomycin susceptible strain V583 grown for 3-7 days and then treated with MMP1. The media was BHI media, protein buffer or MMP1.

### DETAILED DESCRIPTION

The present invention relates to a method of treating a biofilm. More specifically, the invention relates to methods of using matrix metalloproteinase proteins to disrupt or reduce a biofilm, or to prevent the growth of a biofilm on a surface. The present invention also relates to the use of metalloproteinase proteins to treat a biofilm-associated infection in an individual. Thus, a method of the present invention may generally be practiced by contacting a biofilm present on an inanimate or biological surface, with a matrix metalloproteinase protein.

The terminology used herein is for the purpose of describing particular embodiments only and is not intended to be limiting, since the scope of this disclosure will be limited only by the claims.

As used herein and in the appended claims, the singular forms “a,” “an,” and “the” include plural referents unless the context clearly dictates otherwise. For example, a nucleic acid molecule refers to one or more nucleic acid molecules. As such, the terms “a”, “an”, “one or more” and “at least one” can be used interchangeably. Similarly, the terms “comprising”, “including” and “having” can be used interchangeably. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Unless defined otherwise, all technical and scientific terms used herein have the same meaning as commonly understood by one of ordinary skill in the art to which this invention belongs. Although any methods and materials similar or equivalent to those described herein can also be used in the practice or testing of this disclosure, the preferred methods and materials are now described.

One common element of the invention is the use of matrix metalloproteinase proteins, also referred to herein as matrix metalloproteinases, matrix metalloproteases, or “MMPs”. Matrix metalloproteinases are calcium-dependent zinc endopeptidases that are involved in extracellular matrix (ECM) degradation and remodeling of tissues. MMPs are found in vertebrates, invertebrates, and plants, and may be distinguished from other endopeptidases by their dependence on metal ions as cofactors, their ability to degrade ECM, and their specific DNA sequence. Many MMPS are known, including MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. MMPs may be classified into four primary subgroups: collagenases, gelatinases, stromelysins, and film sort (MT)-MMPs. Collagenases (e.g., MMP1, MMP8, MMP13, MMP18) degrade triple helical fibrillary collagens, which are the fundamental segments of bone and ligament. Gelatinases (e.g., MMP2, MMP9) are included in angiogenesis and neurogenesis by corrupting basal lamina molecules and subsequently leading to the cell death. Stromelysins (e.g., MMP3, MMP7, MMP-10, MMP19) are small proteases that degrade segments of the extracellular matrix. MT-MMPs (e.g., MMP14, MMP15, MMP16, MMP17, MMP24, MMP26) activate a few proteases and development components at the cell surface. Any known MMP may be used to practice methods of the disclosure, or to produce compositions of the disclosure. In certain aspects, combinations of MMPS may be used in practicing methods of the invention, or in producing compositions of the invention.

One aspect of the present invention is a method of disrupting or reducing a biofilm, comprising contacting the biofilm with a matrix metalloproteinase protein (MPP). As used herein, the term “biofilm” refers to a population of microorganisms, such as bacteria, that are surrounded by an extracellular matrix generally composed of proteins, polysaccharides, and nucleic acid molecules. The biofilm may comprise any microorganism that is capable of forming or living within a biofilm. The biofilm may comprise a single genus and/or species of microorganism, or it may comprise more than one genus and/or species of microorganism. The microorganism may be a fungus. The microorganism may be a bacteria. The bacteria may be a pathogenic bacteria or a non-pathogenic bacteria. The bacteria may be an aerobic bacteria or an anaerobic bacteria, and may be a Gram-negative bacteria or a Gram-positive bacteria. Bacteria that may be present in the biofilm may come from a genus including, but not limited to, Enterococcus, Streptococcus, Staphylococcus, Bacillus, Lactobacillus, Lactococcus, Escherichia, Pseudomonas, Haemophilus, Klebsiella, and Porphyromonas. Examples of bacteria that may be present in the biofilm include, but are not limited to, Enterococcus faecalis, Escherichia coli, Pseudomonas aeruginosa, Streptococcus pneumonia, Streptococcus viridans, Staphylococcus aureus, Staphylococcus epidermidis, Klebsiella pneumoniae, Haemophilus influenza, Listeria monocytogenes, Lactobacillus plantarum, Lactococcus lactis, Porphyromonas gingivali, and combinations thereof. The biofilm may comprise bacteria from one or more genera selected from the group consisting of Enterococcus, Streptococcus, Staphylococcus, Bacillus, Lactobacillus, Lactococcus, Escherichia, Pseudomonas, Haemophilus, Klebsiella, Porphyromona, and combinations thereof. In one aspect, the biofilm may comprise one or more bacteria selected form the group consisting of Enterococcus faecalis, Escherichia coli, Pseudomonas aeruginosa, Streptococcus pneumonia, Streptococcus viridans, Staphylococcus aureus, Staphylococcus epidermidis, Klebsiella pneumoniae, Haemophilus influenza, Listeria monocytogenes, Lactobacillus plantarum, Lactococcus lactis, Porphyromonas gingivali, and combinations thereof.

As used herein, the terms “disrupting a biofilm” and “reducing a biofilm”, are used interchangeably, and refer to the ability of methods and compositions of the disclosure to degrade one or more components (e.g., protein, polysaccharide) of the biofilm, and/or to kill microorganisms embedded or associated with the biofilm. Disruption or reduction of a biofilm may comprise lessening of the mass of the biofilm by 10 mass % and about 100 mass %, as a result of physical removal of at least part of the biofilm. Disruption or reduction of the biofilm may comprise complete removal of the biofilm.

In such methods, it is understood that the biofilm may be present on a surface. The surface may be an inanimate (i.e., non-biological) surface or it may be a biological surface. Examples of inanimate surfaces include, but are not limited to, a table, a door, a wall, a counter, a handle, a pad, an instrument or tool (e.g., a surgical instrument or tool), or a medical device. Examples of medical devices that may be treated using methods of the disclosure include, but are not limited to, an ultrasound machine, a hemodialysis machine, a contact lens, suture material, a probe, a catheter, a joint prostheses, a bone implant, a dental implant, surgical mesh, a surgical screw or rod, a shunt (e.g., a ventricular shunt), a heart valve, a pacemaker, a defibrillator, or a breast implant.

In such methods, the biofilm may be present on a biological surface. Such biological surfaces include any internal or external part of a body. The biofilm may be present in the bladder, urinary tract, reproductive tract, kidney, heart, middle ear, sinuses, a joint and/or the eye. Examples of biological surfaces on which the biofilm may be located include, but are not limited to, bone, including the medullary cavity, skin, heart tissue, including heart valves, eye tissue, sinus tissue, vascular tissue, airway tissue, lung tissue, mucous membranes, oral tissue, teeth, and sinus tissue. The biofilm may be present in a wound. Such wounds include, but are not limited to, incisions, lacerations, avulsions, abrasions, punctures, and burns.

A MMP used in methods of the disclosure may be any known MMP, as long as it is capable of degrading at least one component in a biofilm. The MMP may be from a plant, an invertebrate or a vertebrate. The MMP may be from a mammal, such as a human. The MMP may be selected from the group consisting of MMP1, MMP2. MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. The MMP may be a gelatinase, such as MMP2 or MMP9. The MMP may be a collagenase, such as MMP1, MMP8, MMP13 or MMP18. The MMP may be a stromelysin. The MMP may be a film-sort MMP. In certain methods of the disclosure, more than one MMP may be contacted with the biofilm.

In such methods, contact of an MMP with the biofilm may comprise contacting a therapeutic composition containing a therapeutically effective amount of the MMP, with the biofilm. A therapeutic compositions of the disclosure comprises a pharmaceutically acceptable carrier. The term “pharmaceutically acceptable carrier” refers to a diluent or vehicle with which the MMP is administered. Carriers are acceptable in the sense of being compatible with other ingredients in the composition, and in that they are not deleterious to the recipient thereof. Such pharmaceutically acceptable carriers may include, but are not limited to, sterile liquids, such as water, oil, polyethylene glycol, saline solutions, dextrose solutions, and glycerol solutions. Therapeutic compositions of the present invention may also include other ingredients, such as buffers, or stabilizers. Therapeutic composition of the disclosure may be formulated as a liquid solution, a buffered solution, a paste, a slurry, a gel, a lotion, an emollient, a spray, a rinse, or an emulsion.

As used herein, a “therapeutically effective amount” refers to an amount of MMP that, when administered to an individual being treated for a biofilm, alleviates at least one symptom of a disease or disorder resulting from presence of the biofilm.

In addition to a MMP, a pharmaceutically acceptable composition of the disclosure may comprise additional agents for disrupting or treating a biofilm. Such agents may include, but are not limited to, dispersants, surfactants, detergents, enzymes other than MMPs, antimicrobial agents, biocides, and chelating agents.

One aspect of the present invention is a method of inhibiting the growth of a microorganism, the method comprising contacting the microorganism with a matrix metalloproteinase. The microorganism may be a bacteria. The bacteria may be a pathogenic bacteria or a non-pathogenic bacteria. The bacteria may be an aerobic bacteria or an anaerobic bacteria, and may be a Gram-negative bacteria or a Gram-positive bacteria. Bacteria that may be present in the biofilm may come from a genus including, but not limited to, Enterococcus, Streptococcus, Staphylococcus, Bacillus, Lactobacillus, Lactococcus, Escherichia, Pseudomonas, Haemophilus, Klebsiella, Porphyromonas, and combinations thereof. Examples of bacteria that may be present in the biofilm include, but are not limited to, Enterococcus faecalis, Escherichia coli, Pseudomonas aeruginosa, Streptococcus pneumonia, Streptococcus viridans, Staphylococcus aureus, Staphylococcus epidermidis, Klebsiella pneumoniae, Haemophilus influenza, Listeria monocytogenes, Lactobacillus plantarum, Lactococcus lactis, Porphyromonas gingivali, and combinations thereof. Thus, in one aspect, the biofilm may comprise bacteria from one or more genera selected from the group consisting of Enterococcus, Streptococcus, Staphylococcus, Bacillus, Lactobacillus, Lactococcus, Escherichia, Pseudomonas, Haemophilus, Klebsiella, Porphyromona, and combinations thereof. In one aspect, the biofilm may comprise one or more bacteria selected form the group consisting of Enterococcus faecalis, Escherichia coli, Pseudomonas aeruginosa, Streptococcus pneumonia, Streptococcus viridans, Staphylococcus aureus, Staphylococcus epidermidis, Klebsiella pneumoniae, Haemophilus influenza, Listeria monocytogenes, Lactobacillus plantarum, Lactococcus lactis, Porphyromonas gingivali, and combinations thereof.

The microorganism may be present on a surface. The surface may be an inanimate (i.e., non-biological) surface, such as, a table, a door, a wall, a counter, a handle, a pad, an instrument or tool (e.g., a surgical instrument or tool), or a medical device. Examples of medical devices that may be treated using methods of the disclosure include, but are not limited to, an ultrasound machine, a hemodialysis machine, a contact lens, suture material, a probe, a catheter, a joint prostheses, a bone implant, a dental implant, surgical mesh, a surgical screw or rod, a shunt (e.g., a ventricular shunt), a heart valve, a pacemaker, a defibrillator, or a breast implant.

In such methods, the microorganism may be present on a biological surface. Such biological surfaces include any internal or external part of a body. The microorganism may be present in the bladder, urinary tract, reproductive tract, kidney, heart, middle ear, sinuses, a joint and/or the eye. Examples of biological surfaces on which the microorganism may be located include, but are not limited to, bone, including the medullary cavity, skin, heart tissue, including heart valves, eye tissue, sinus tissue, vascular tissue, airway tissue, lung tissue, mucous membranes, oral tissue, teeth, and sinus tissue. The microorganism may be present in a wound. Such wounds include, but are not limited to, incisions, lacerations, avulsions, abrasions, punctures, and burns.

A matrix metalloproteinase protein (MMP) used in methods of the disclosure may be any known MMP, as long as it is capable of inhibiting the growth of an undesired microorganism. The MMP may be from a plant, an invertebrate or a vertebrate. The MMP may be from a mammal, such as a human. The MMP may be selected from the group consisting of MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. The MMP may be a gelatinase, such as MMP2 or MMP9. The MMP may be a collagenase, such as MMP1, MMP8, MMP13 or MMP18. The MMP may be a stromelysin. The MMP may be a film-sort MMP. In certain methods of the disclosure, more than one MMP may be contacted with the microorganism.

In such methods, contact of an MMP with the biofilm may comprise contacting a therapeutic composition containing a therapeutically effective amount of the MMP, with the microorganism.

Because biofilms protect the resident microorganisms from antimicrobial agents, their presence often results in disease due to the inability of the body to eliminate the pathogenic microorganisms. It will be appreciated by those skilled in the art that disrupting or reducing the biofilm, and/or inhibiting growth of biofilm-associated microorganisms, would be beneficial to eliminating the microorganism and reducing disease. Thus, one embodiment of the invention is a method of treating a biofilm-associated disease or disorder in an individual, the method comprising administering to the individual a therapeutic composition comprising a therapeutically effective amount of a matrix metalloproteinase (MMP), so that the MMP comes in contact with the biofilm.

As used herein, the term “treating” means administering a therapeutic composition of the disclosure to an individual, wherein the amount of MMP in the composition has been shown to alleviate or reduce at least one symptom in other patients having the same biofilm-associated infection disease or disorder, as the individual being treated. The treatment may alleviate, reduce, or eliminate at least one symptom related to the presence of the biofilm. Such treatment may disrupt or reduce the biofilm in the individual. The treatment may result in complete elimination of the biofilm from the individual. The treatment may inhibit the growth of, or kill, microorganisms in the biofilm in the individual.

The terms individual, patient, and subject are well-recognized in the art, and are herein used interchangeably to refer to any human or other animal susceptible to biofilm-associated disease. Examples include, but are not limited to, humans and other primates, including non-human primates such as chimpanzees and other apes and monkey species; farm animals such as cattle, sheep, pigs, seals, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The terms individual, subject, and patient by themselves, do not denote a particular age, sex, race, and the like. Thus, individuals of any age, whether male or female, are intended to be covered by the present disclosure and include, but are not limited to, the elderly, adults, children, babies, infants, and toddlers. Likewise, the methods of the present invention can be applied to any race, including, for example, Caucasian (white), African-American (black), Native American, Native Hawaiian, Hispanic, Latino, Asian, and European. An infected subject is a subject that is known to have biofilm-associated disease or disorder.

In such methods, the disease or disorder may be any disease or disorder, signs and/or symptoms of which are due to the presence of a biofilm in the individual. Examples of such disease and disorders include, but are not limited to periodontal disease (gum disease, tooth decay), infection and/or inflammation of tissue such as a wound, otitis media, prostatitis, pneumonia, lung infection, meningitis, vaginosis, urinary tract infection, conjunctivitis, sinusitis, atherosclerosis, endocarditis, leptospirosis, osteomyelitis, and infection of a medical device, such as a catheter.

In such methods, the biofilm may be present on any internal or external surface of the individual's body. The biofilm may be present in the bladder, urinary tract, reproductive tract, kidney, heart, middle ear, sinuses, a joint and/or the eye. The biofilm may be present on, bone, including the medullary cavity; on skin; on heart tissue, including heart valves; on eye tissue; on sinus tissue; on vascular tissue; on airway tissue; on lung tissue; on mucous membranes; on oral tissue; on teeth; or on sinus tissue. The biofilm may be present in a wound, such as an incision, a laceration, an avulsion, an abrasions, a puncture, or a burn.

Administration of the therapeutic composition to the individual can be achieved using any suitable method. For example, if the biofilm is on the skin, the therapeutic composition may be applied directly to the region containing the biofilm. Likewise, biofilm-containing wounds may be treated by direct application of the therapeutic composition to the wound. In such methods, the therapeutic composition may be administered as a liquid, or as a paste, a slurry, a gel, a lotion, an emollient, a spray, a rinse, or an emulsion. If the biofilm is present in an internal cavity, such as the sinuses, administration may be best achieved by formulating the therapeutic composition in a liquid formulation, such as a spray or rinse, although pastes, creams, gels, and the like, are not excluded from such a method. If the biofilm is present in an internal cavity not easily accessible, administration may comprise surgical access, which includes laparoscopic surgery and lavage of internal cavities.

In such methods, the MMP may be any known MMP, as long as it is capable of degrading at least one component in the biofilm. The MMP may be from a plant, an invertebrate or a vertebrate. The MMP may be from a mammal, such as a human. The MMP may be a gelatinase, such as MMP2 or MMP9. The MMP may be a collagenase, such as MMP1, MMP8, MMP13 or MMP18. The MMP may be a stromelysin. The MMP may be selected from the group consisting of MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. In such methods, the therapeutic composition may comprise more than one MMP.

In such methods, the therapeutic composition may comprise additional agents for disrupting or treating the biofilm. Such agents may include, but are not limited to, dispersants, surfactants, detergents, enzymes other than MMPs, antimicrobial agents, biocides, and chelating agents.

In such methods, the individual may already be receiving treatment (e.g., antibacterial treatment) for the biofilm-associated disease or disorder, and a therapeutic composition of the present disclosure is administered to enhance treatment of the disease or disorder. Thus, one aspect of the invention is a method to enhance treatment of a biofilm-associated disease or disorder in an individual being treated for such a disease or disorder, comprising administering to the individual a therapeutic composition comprising a therapeutically effective amount of a matrix metalloproteinase (MMP), so that the MMP comes in contact with the biofilm. Such a treatment may reduce the mass of the biofilm, thereby allowing greater access of the antibacterial treatment into the biofilm, where it can contact the biofilm-associated bacteria.

One aspect of the invention is a therapeutic composition comprising a therapeutically effective amount of an MMP, suitable for administration to an individual having a biofilm-associated disease or disorder. The MMP may be any known MMP, as long as it is capable of degrading at least one component in the biofilm. The MMP may be from a plant, an invertebrate or a vertebrate. The MMP may be from a mammal, such as a human. The MMP may be a gelatinase, such as MMP2 or MMP9. The MMP may be a collagenase, such as MMP1, MMP8, MMP13 or MMP18. The MMP may be a stromelysin. The MMP may be selected from the group consisting of MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. The therapeutic composition may comprise more than one MMP. The therapeutic composition may comprise a pharmaceutically acceptable carrier, such as sterile liquids, such as water, oil, polyethylene glycol, saline solutions, dextrose solutions, and glycerol solutions. The therapeutic composition may also include other ingredients, such as buffers, stabilizers, dispersants, surfactants, detergents, enzymes other than MMPs, antimicrobial agents, biocides, and chelating agents. In one aspect, the therapeutic composition comprises a MMP and an antibiotic, such as trimethoprim-sulfamethoxazole, clindamycin, tetracycline, doxycycline, or linezolid. The therapeutic composition may be formulated in a liquid form, or as a paste, a slurry, a gel, a lotion, an emollient, a spray, a rinse, or an emulsion.

One aspect of the invention is a method of preventing biofilm formation, comprising applying to the surface of an item an amount of a MMP effective to prevent the formation of the biofilm. The MMP may be any known MMP, as long as it is capable of degrading at least one component in the biofilm. The MMP may be from a plant, an invertebrate or a vertebrate. The MMP may be from a mammal, such as a human. The MMP may be a gelatinase, such as MMP2 or MMP9. The MMP may be a collagenase, such as MMP1, MMP8, MMP13 or MMP18. The MMP may be a stromelysin. The MMP may be selected from the group consisting of MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. More than one MMP may be applied to the item. The item may be a medical device, which may be an ultrasound machine, a hemodialysis machine, a contact lens, suture material, a probe, a catheter, a joint prostheses, a bone implant, a dental implant, surgical mesh, a surgical screw or rod, a shunt (e.g., a ventricular shunt), a heart valve, a pacemaker, a defibrillator, or a breast implant. In such methods, more than one MMP may be applied to the item. In such a method, the effective amount of MMP may be applied as a composition that may be a liquid, a buffered solution, a paste, a slurry, a gel, a lotion, an emollient, a spray, a rinse, or an emulsion.

One aspect of the invention is an item, such as a medical device, tubing, a cannula, a probe, a catheter, a joint prostheses, a bone implant, a dental implant, surgical mesh, a surgical screw or rod, a shunt (e.g., a ventricular shunt), a heart valve, a pacemaker, a defibrillator, or a breast implant, comprising on at least one surface, an amount of MMP sufficient to prevent the formation of a biofilm. In one aspect, the item may be wound dressing, such as gauze, a hydrocolloid dressing, a hydrogel dressing, or an alginate dressing. The MMP may be any known MMP, as long as it is capable of degrading at least one component in the biofilm. The MMP may be from a plant, an invertebrate or a vertebrate. The MMP may be from a mammal, such as a human. The MMP may be a gelatinase, such as MMP2 or MMP9. The MMP may be a collagenase, such as MMP1, MMP8, MMP13 or MMP18. The MMP may be a stromelysin. The MMP may be selected from the group consisting of MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. The item may comprise more than one MMP.

One aspect of the invention is a method of inhibiting the growth of a microorganism, the method comprising contacting the organism with a matrix metalloproteinase (MMP).

One aspect of the invention is a kit for practicing methods of the disclosure, wherein the kit comprises at least one MMP intended for use in disrupting or reducing a biofilm, preventing the formation of a biofilm on an item, or treating an individual for a biofilm-associated disease or disorder. The kit may comprise instructions for using the MMP to disrupt or reduce a biofilm, prevent the formation of a biofilm on an item, inhibit the growth of a microorganism, or treat an individual for a biofilm-associated disease or disorder. The kit may comprise any known MMP, as long as it is capable of degrading at least one component in the biofilm. The MMP may be from a plant, an invertebrate or a vertebrate. The MMP may be from a mammal, such as a human. The MMP may be a gelatinase, such as MMP2 or MMP9. The MMP may be a collagenase, such as MMP1, MMP8, MMP13 or MMP18. The MMP may be a stromelysin. The MMP may be selected from the group consisting of MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, and MMP28. The item may comprise more than one MMP. The MMP may be in a liquid solution, a buffered solution, a paste, a slurry, a gel, a lotion, an emollient, a spray, a rinse, or an emulsion. The kit may comprise a therapeutic composition for treating an individual for a biofilm-associated disease or disorder. In addition, the kit may comprise tubes, syringes, needles, vials, diluents, and the like, for practicing methods of the disclosure.

The following examples are put forth so as to provide those of ordinary skill in the art with a complete disclosure and description of how to make and use the embodiments, and are not intended to limit the scope of what the inventors regard as their invention nor are they intended to represent that the experiments below are all or the only experiments performed.

### EXAMPLES

### Example 1. Purification of Matrix Metalloproteinase 1 (MMP1)

Active MMP1 was purified as described by Kumar et al. (Science 2004; 306(5693):108011). Briefly, the cDNA sequence of MMP1 was optimized for expression in E. coli and inserted into a pET-21b (+) vector between the Ndel (N-terminal) and HindIII (C-terminal) restriction sites. The plasmid was transformed into Rosetta (DE3) pLysS competent cells (Novagen, 70956), and the cells cultured in Luria Broth media (Sigma, L3022) at 37° C. and 250 rpm, in presence of chloramphenicol (34 ug.ml) and ampicillin (100 ugml), until the optical density reached OD600=0.1. The cells were induced for 5 hours (hrs) with 1 mM Isopropyl β-D-1-thiogalactopyranoside (IPTG) and harvested by centrifugation. 1 gram (g) of the centrifuged cells was reconstituted in 7 ml of lysis buffer (50 mM Tris base (Sigma, T4661), pH 9.0, 100 mM NaCl (Sigma, S9888), 200 uM ZnCl2 (Sigma, 208086), 400 uM CaCl2 (Sigma, 746495), freshly prepared 1% Triton X-100 (Sigma, T8787), 0.1 mg/ml trypsin (Worthington, TPCK-treated and irradiated, LS003750), and 1 mg/ml lysozyme (Sigma, L6878). The reconstituted cells were incubated for 18 hrs at 37° C. at 250 rpm and centrifuged to collect the supernatant, followed by centrifugation using 30 kD cut-off filters. MMP1 was quantified using Bradford assay and analyzed by SDS PAGE (data not shown).

### Example 2. Inhibitory Effect of MMP-1 on Biofilms

To quantify the ability of MMPs to inhibit biofilm formation, biofilms were grown in the presence of MMP1. Briefly, single colonies of E. faecalis cells were grown for 18 hrs with 250 rpm shaking at 37° C. in 5 ml of BHI media (BD, BBL Brain Heart Infusion Broth, 211059). To prepare log-phase cultures for inoculation, strains were subcultured in microtiter plates (Thermo Scientific, 96-well flat bottom sterile with lid and untreated, 266120) by adding 20 μl of overnight culture to 180 μl of BHI broth and incubated for 5 hr with 150 rpm shaking at 37° C. to obtain OD600 ˜0.5. For inhibition assays, 20 μl of OD ˜0.5 bacterial inoculum was added to 50 μl of 1 mg/ml MMP1 and 180 μl of BHI media in each well. The plates were incubated at 37° C., and every 24 hours the solution in each well aspirated, and 50 μl of fresh MMP1 (1 mg/ml) and 200 μl of BHI media were added to each well. After the desired duration of incubation (1 to 7 days) to allow formation of a biofilm, the solution was aspirated from the wells, each well washed with PBS buffer (Sigma, P3813, pH 7.4) to remove unbound planktonic cell, and the bound cells stained with 300 μl of 0.1% crystal violet (CV) stain (Sigma, C0775) for 15 min. The stain was removed and each cell washed three times with 300 μl of PBS buffer, after which the biofilm-associated CV was removed from the biofilm by washing each cell with 30% acetic acid (Sigma, A6283). The absorbance of CV stain dissolved in acetic acid was measured at 635 nm using a plate reader (Biotek, Synergy2-Cam4, Software-Gen5-1.08). To determine the baseline activity of the CV assay, experiments with only BHI media were performed.

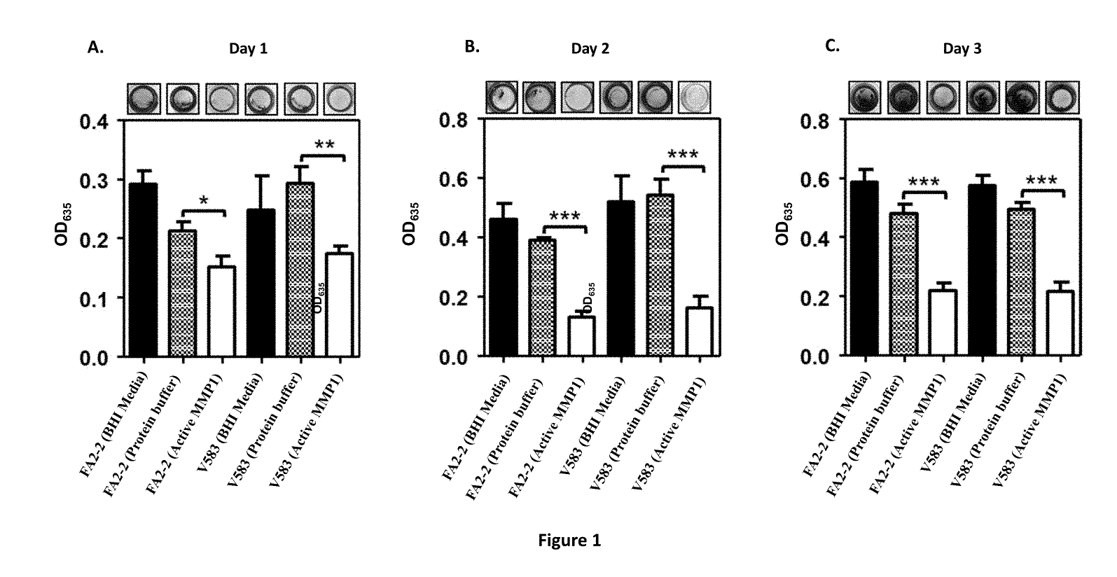

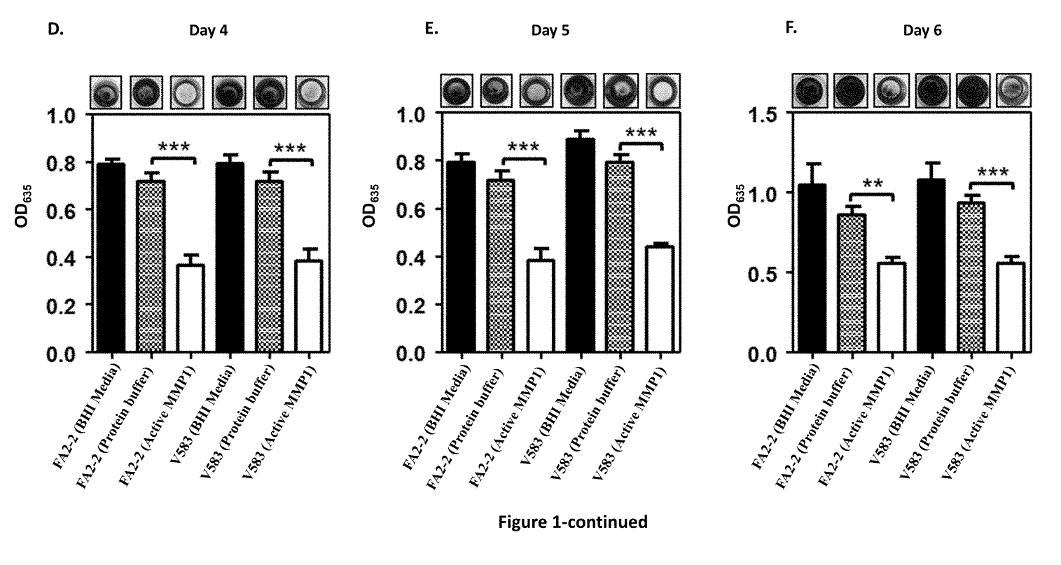

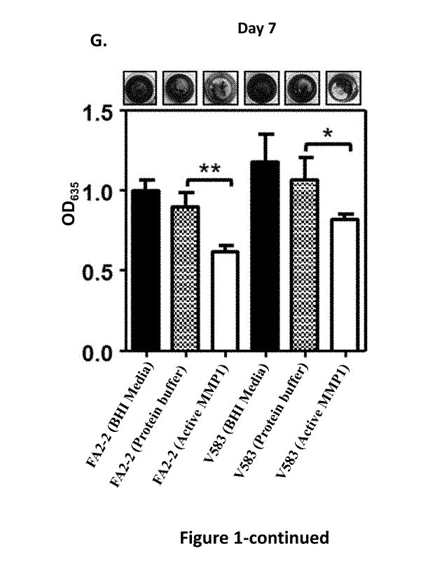

The results of this assay are show in FIG. 1, which illustrates the results of seven day biofilm growth by E. faecalis strains FA2-2 (vancomycin susceptible) and V583 (vancomycin resistant). The images of the wells captured by a camera (shown above the graph in FIGS. 1A-G) clearly show the inhibition effect, even for 7-day-old (FIG. 1G) inhibition biofilms. While the Triton X-100 in the protein buffer had a small effect on the biofilms, in all cases the effect of protein buffer plus MMP1 showed a more significant effect on the biofilm formation for both strains. Before dissolving CV stain in acetic acid, imaging of wells indicated visible reduction in bacterial biomass after MMP1 treatment as a qualitative indicator of inhibition.

### Example 3. Disruptive Effect of MMP-1 on Biofilms

It is likely that biofilm forming pathogens would already form a biofilm by the time of an accurate clinical diagnosis. Therefore, an ideal anti-biofilm agent should also be able to effectively disrupt the established biofilms. Hence, we quantified the effect of MMP1 on established biofilms of E. faecalis strains To quantify the ability of MMP1 to disrupt a biofilm, 1-7 day old biofilms were grown in 96-well plates, as described in Example 2. After biofilms were established for the desired duration, 50 μl of 1 mg/ml MMP1 was added to each well, and the plate incubated for 24 hr at 37° C. without shaking. MMP1-treated biofilms were then quantified using the CV assay described in Example 2. Results of quantitative CV assay was qualitatively confirmed by images of wells stained with crystal violet. Effects of MMP1 were compared with two control experiments with BHI media and protein buffer containing 1% Triton X-100.

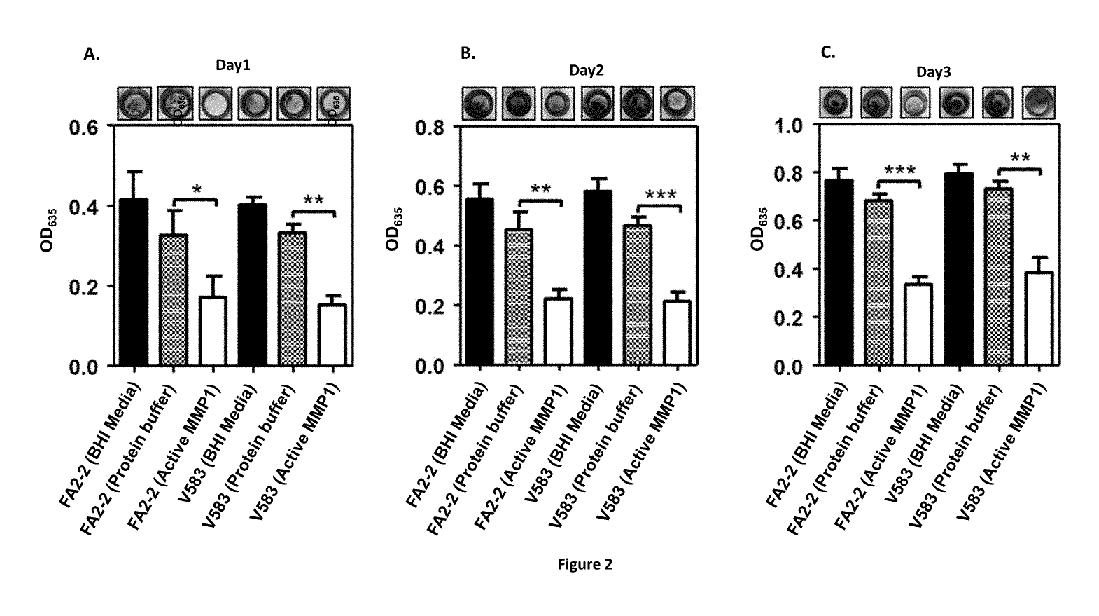

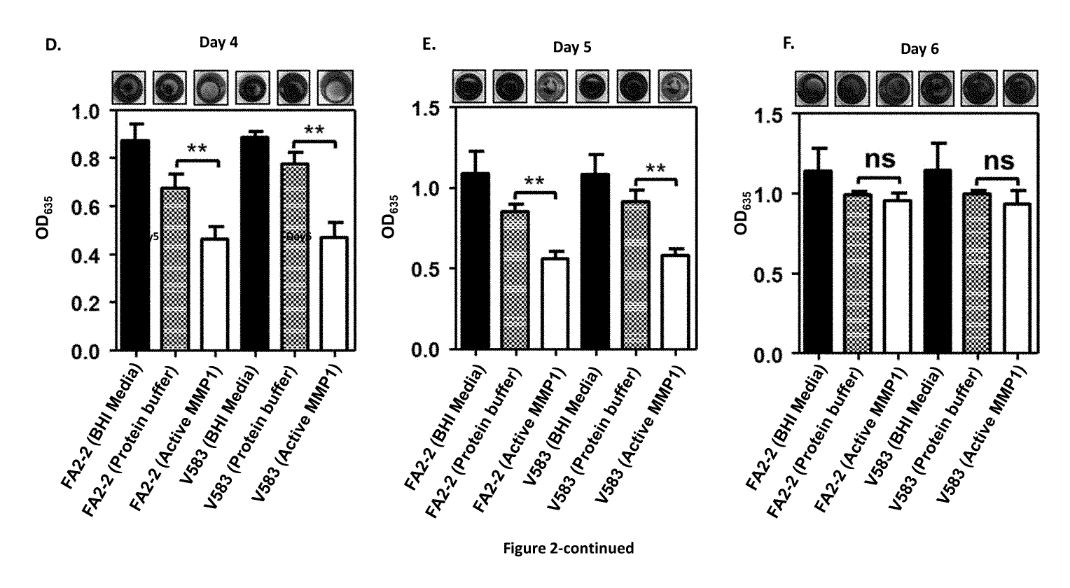

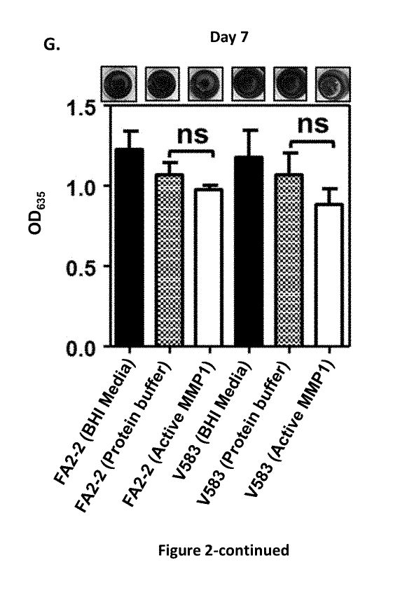

The results, shown in FIGS. 2A-G, demonstrate that MMP1 effectively disrupted 1-day-old to 5-day-old biofilms (FIGS. 2A-E). However, 6-day-old and 7-day-old biofilms (FIGS. 2F-G) were not completely disrupted by MMP1.

### Example 4. Scanning Electron Microscope (SEM) Imaging of Established Biofilms

To investigate changes in biofilm architecture after treatment with MMP1 for 24 hr, biofilms were grown on plastic coverslips. Briefly, 300 μl of log-phase culture inoculation was added to 5 ml BHI media in wells of untreated 6-well plate (Celltreat Scientific Products, 229506). Biofilms were grown for 3 to 7 days by immersing 22 mm×22 mm plastic coverslips (Carolina Biological Supply Company, 632900) in the solution. To study the effect of MMP1, 500 μl of 1 mg/ml MMP1 was added in each well. For control experiments, 500 μl of BHI media and protein buffer were added instead of MMP1. To image the biofilms, the solution was aspirated and coverslips washed three times with 500 μl of sterile PBS and dried at 37° C. for 24 hrs. Environmental SEM was done using a Phenom Pro-Scanning Electron microscope without extra sample preparation and high vacuum necessary for conventional electron microscopy. This approach allowed for observation of changes in the extracellular polymeric substances (EPS) caused by sample processing.

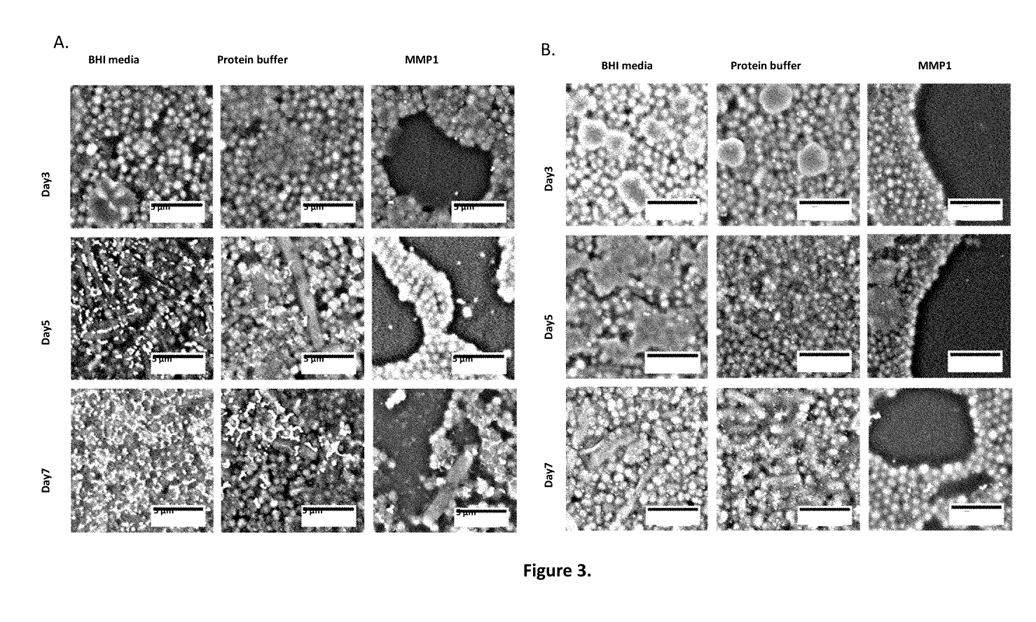

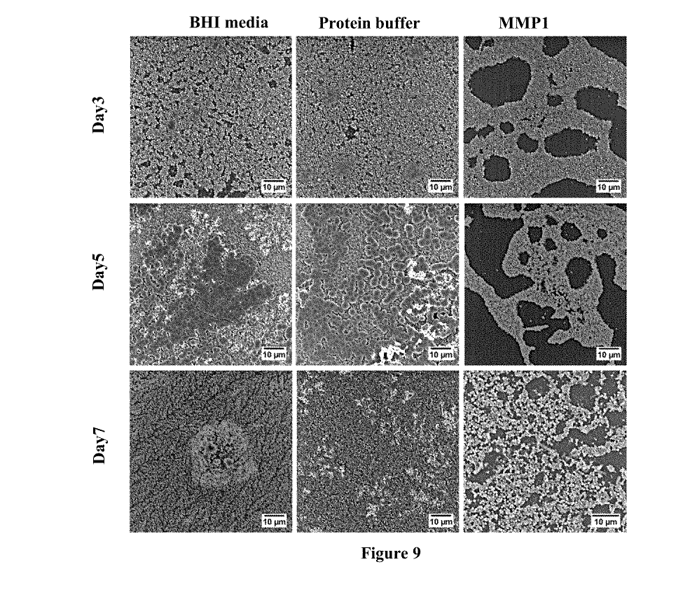

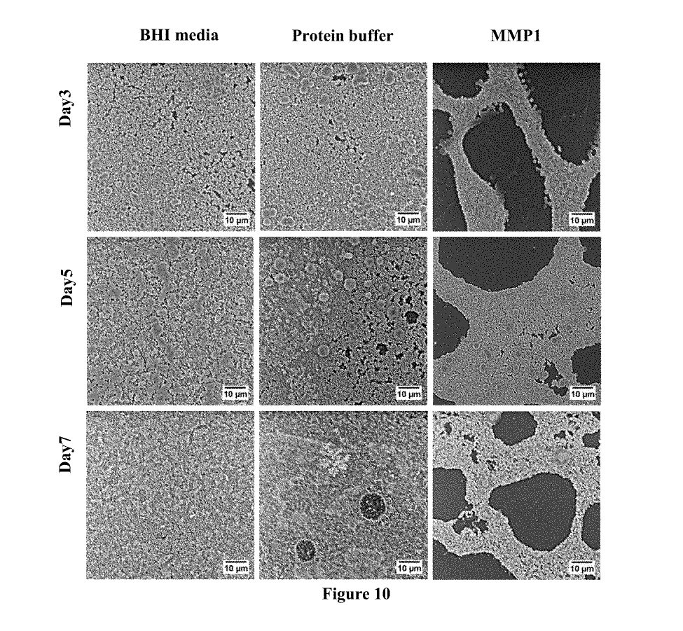

The results of this study are shown in the FIGS. 3A and 3B. MMP1-treated biofilms showed less bacterial colonization and high disruption in biofilm architecture as compared to the control biofilms (FIGS. 3A and 3B; FIGS. 9 and 10). SEM images of 3-day-old biofilms without MMP1 treatment showed uniform layers of biofilm, whereas MMP1-treated biofilms showed a sporadic layer with patches of cells and large areas of clearance. Similar disruption of 5-day-old biofilms were observed after MMP1 treatment. For 7-day-old biofilms, MMP1 treatment did not completely destabilized the biofilm and thin layers of cells with lesser amounts of observable biofilm matrix were observed. The extended structures in SEM images (FIGS. 3A and 3B) were not due to bacterial contamination because controls with only media did not lead to any growth over the course of experiments and similar structural features have been reported before for E. faecalis biofilms.

### Example 5. Confocal Laser Scanning Analysis of Biofilms

Neither CV assays nor SEM informs whether MMP1 has an impact on bacterial viability. To perform live/dead assays and check the viability of bacteria, biofilms were grown on plastic coverslips in the presence of MMP1 and imaged by confocal laser scanning microscopy (CLSM) after staining with acridine orange and propidium iodide Briefly, biofilms were grown in the presence of MMP1 as described for SEM imaging. After brief air drying, biofilms were immediately stained with 500 μl of 10 μg/ml solution of propidium iodide (Thermo Fisher Scientific, Invitrogen, P3566) and 10 μg/ml acridine orange for 2 min. Coverslips were washed three times with 1 ml deionized water to remove unbound stain. Confocal laser scanning microscopic imaging was performed with a confocal microscope (Olympus, FV10i). Sensitivity was set at 40% for both the lasers. Images were processed using Fiji ImageJ software to merge green (live cells) and orange fluorescence (dead cells). Cells with compromised membranes stained red/orange, whereas viable bacteria with intact cell structure stained green.

The results show the presence of both live and dead bacteria in varying amounts in 3-day-old to 7-day-old biofilms (data not shown). For 3-day-old biofilms, more viable bacterial cells were observed than dead cells. For older biofilms, less viable cells were observed for both MMP1-treated and control biofilms as expected. No consistent pattern of live/dead cells was observed for different experimental conditions arising due to the compounding effects of deaths caused due to natural life cycle, Triton X100 in protein buffer, potential antibacterial effect of MMP1 and sample preparation for confocal imaging. Additionally, MMP1-treated biofilms had less biomass because cells were not able to attach and form biofilms. The control protein buffer containing Triton X-100 had a measurable effect especially at day 3, which disappeared at later stages. Triton X-100 is a known antimicrobial detergent against Gram-positive and Gram-negative bacteria and lyse the bacterial cells by targeting bacterial membranes. Therefore, Triton X-100 affects thinner biofilms at the early stage. However, older biofilms become thick enough to prevent Triton X-100 from penetrating the biofilms and killing the resident bacteria. Triton X-10, which is present in MMP1 purification buffer, is known to stabilize proteins.

### Example 6. Quantification of Viable Cells in Inhibition and Disruption of Biofilms

While confocal microscopy showed that biofilms contained both live and dead bacteria after MMP1 treatment, a consistent pattern was not observed. Thus, a CFU assay was performed to quantify only the live cells. Briefly, a standard plate count assay was performed. Biofilms (day 1 to day 7) were incubated with MMP1, and planktonic cells were washed with PBS buffer. After removing planktonic cells, cells contained within biofilms were scrapped using a sterile tip and suspended in 100 μl of PBS buffer. A series of 10-fold dilutions were prepared and 100 μl of final dilutions were spread onto BHI plates using a sterile L-shaped spreader. CFUs were determined by counting the bacterial colonies on BHI plates. The total CFU counts were converted to Log₁₀CFU and plotted. Similar experiments were conducted in which a catalytically inactive MMP1, having a point mutation at E219Q, was used to confirm that the observed activities on biofilms were indeed due to MMP1 activity. (The E219Q mutation has been shown to inhibit MMP1 activity on collagen, a well-known substrate for MMP1.)

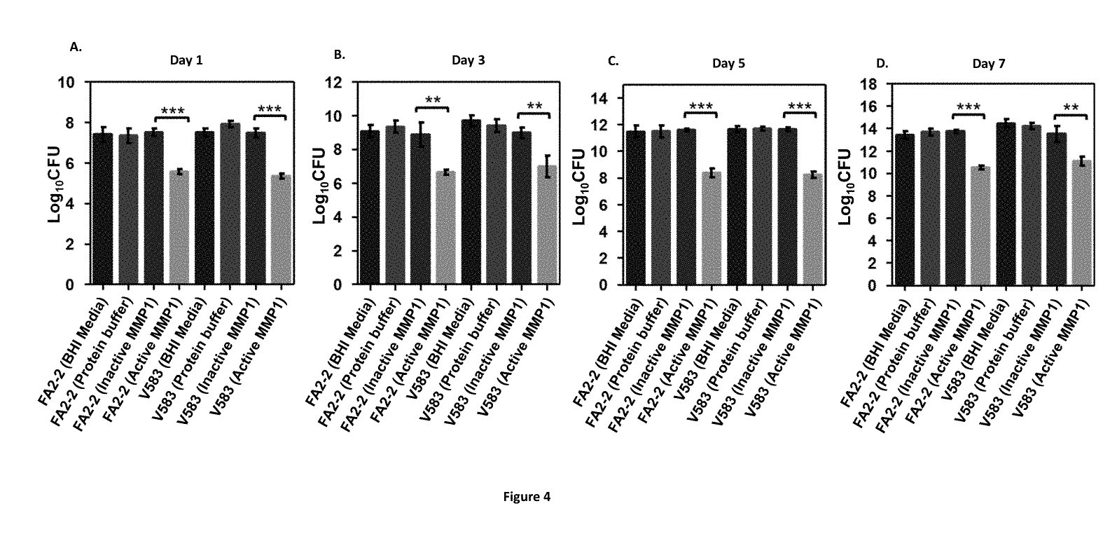

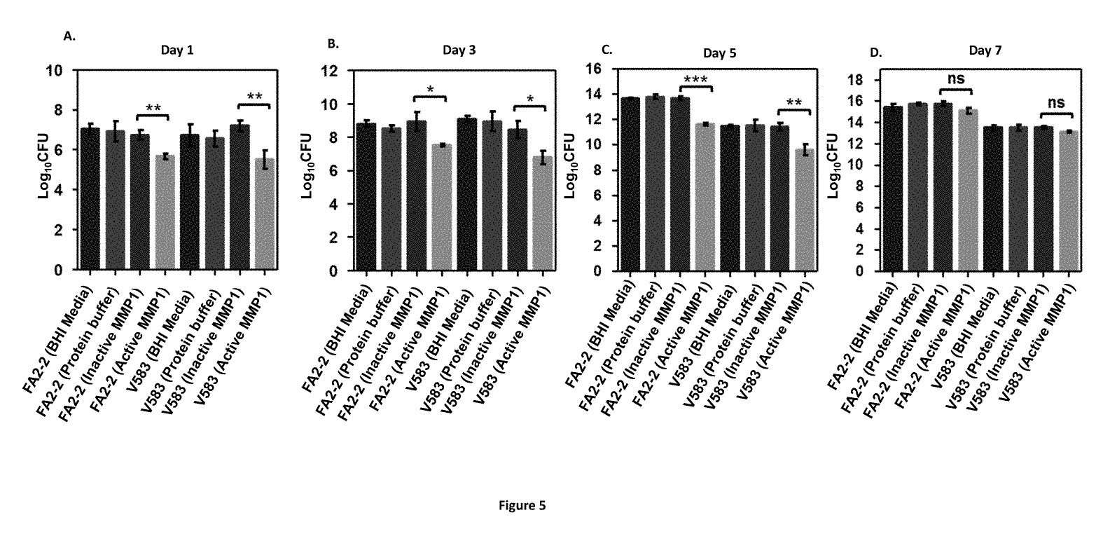

The results of these analyses are shown in FIGS. 4A-D and 5A-D. The CFU assays showed that active MMP1 clearly inhibited biofilm growth up to 7 days for both strains, as compared to control experiments with BHI media, protein buffer, and inactive MMP1. However, disruption of 7-day-old biofilm was not significant.

### Example 7. Analysis of Overall Biofilm Integrity Following MMP Treatment

Since MMP1 is a protease, it was postulated that MMP1 inhibits and disrupts the overall biofilm structure by degrading proteins in biofilms. To test this hypothesis, a Congo red assay was performed to evaluate degradation of the overall biofilm structure by MMP1. Both inhibition and disruption effects were studied. Briefly, after incubation of biofilms with MMP1, the solution was aspirated from wells. Biofilms were then washed with PBS buffer (Sigma, P3813, pH 7.4) to remove unbound planktonic cells. Staining was performed using 300 μl of 0.1% Congo red Hi Cert/ACS stain (Himedia, GRM508-10G) for 24 hrs, followed by washing three times with 300 μl of PBS buffer. After washing, Congo red stain bound to biofilms was dissolved in DI water, and absorption measured at 490 nm using a plate reader.

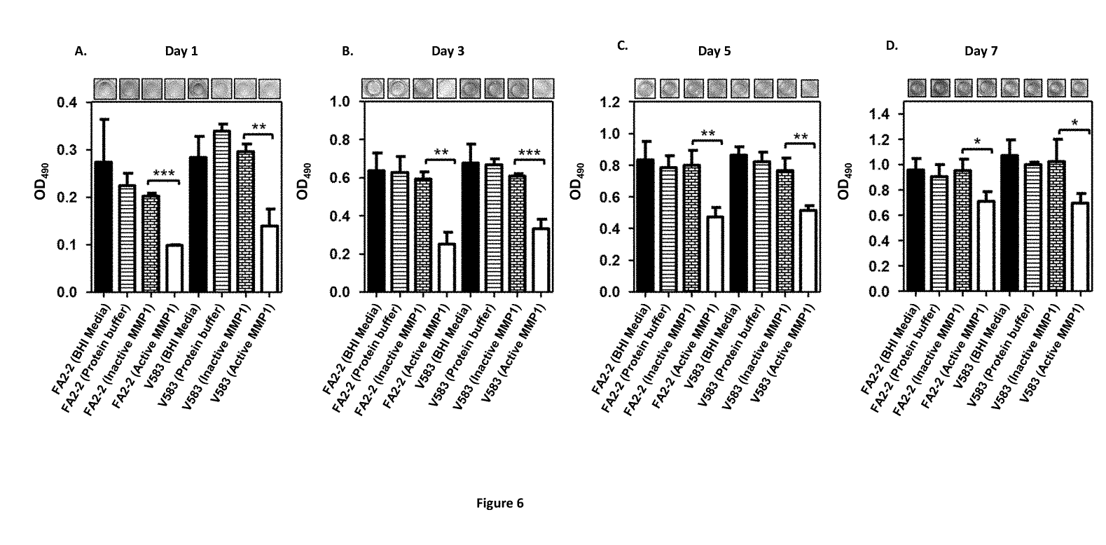

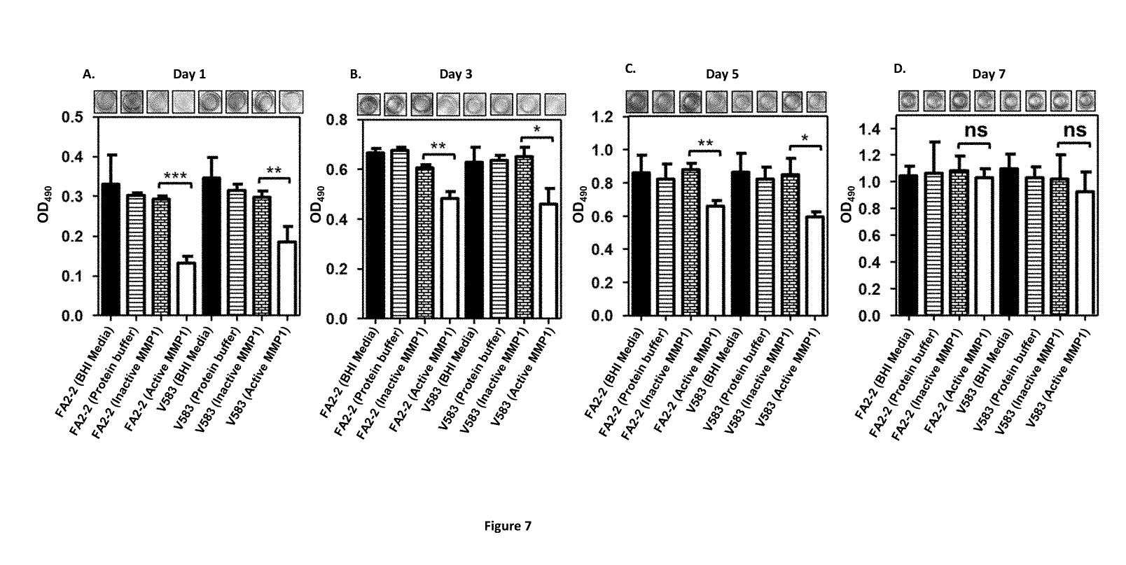

The results, shown in FIGS. 6A-D & 7A-D, revealed that active MMP1 significantly degraded proteins in biofilms, while catalytically inactive MMP1 showed no significant degradation.

### Example 8. Effect of MMP1 on Planktonic Growth

This Example demonstrates the effect of MMP1 on bacterial cell growth. Briefly, log-phase cultures were grown as previously described. 10 μl of log-phase culture was inoculated in 200 μl of sterile BHI broth in 96-well microtiter plate, and 50 μl of 1 mg/ml MMP1 was added to obtain a total reaction volume of 260 μl. Absorption measurements at 600 nm were done every 30 min at 37° C., over a period of 8 hours, using a plate reader. For control experiments BHI broth or protein buffer were used instead of MMP1.

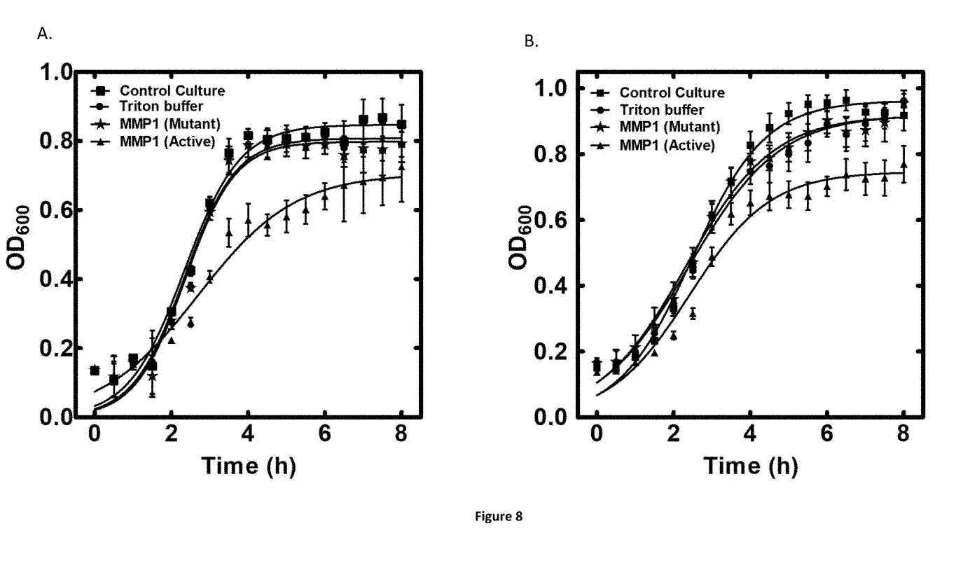

As shown in FIGS. 8A & 8B, optical density measured by absorption at 600 nm (OD₆₀₀) was significantly decreased in MMP1-treated samples after 2 hrs compared to controls. Maximum difference was observed after 4-6 hrs of incubation. The results clearly showed that MMP1 treatment significantly reduced the growth of E. faecalis cells.

Ranges have been discussed and used within the forgoing description. One skilled in the art would understand that any sub-range within the stated range would be suitable, as would any number within the broad range, without deviating from the invention.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

What is claimed:

1. A method of disrupting a biofilm, comprising contacting the biofilm with a therapeutic agent comprising a matrix metalloprotease protein (MMP).

2. The method of claim 1, further comprising reducing the mass of the biofilm.

3. The method of claim 1, wherein the biofilm is present on at least one surface of an inanimate object.

4. The method of claim 3, wherein the inanimate object is a medical device.

5. The method of claim 1, wherein the biofilm is present on at least one biological surface.

6. The method of claim 1, wherein the MMP is selected from the group consisting of MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28, and combinations thereof.

7. The method of claim 1, wherein the MMP is a gelatinase.

8. The method of claim 1, wherein the biofilm is a bacterial biofilm.

9. The method of claim 1, wherein the biofilm comprises a bacteria from a genus selected form the group consisting of Enterococcus, Streptococcus, Staphylococcus, Bacillus, Lactobacillus, Lactococcus, Escherichia, Pseudomonas, Haemophilus, Klebsiella, Porphyromona, and combinations thereof.

10. A method of treating an individual for a biofilm-related disease or disorder, comprising:
administering to the individual a therapeutic composition comprising a therapeutically effective amount of a matrix metalloproteinase protein (MMP), wherein the MMP contacts the biofilm; and
reducing a mass of the biofilm.

11. The method of claim 10, wherein the MMP is selected from the group consisting of MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28, and combinations thereof.

12. The method of claim 1, wherein the MMP is a gelatinase.

13. The method of claim 10, wherein the biofilm is a bacterial biofilm.

14. The method of claim 10, wherein the biofilm comprises a bacteria from a genus selected form the group consisting of Enterococcus, Streptococcus, Staphylococcus, Bacillus, Lactobacillus, Lactococcus, Escherichia, Pseudomonas, Haemophilus, Klebsiella, Porphyromona, and combinations thereof.

15. A therapeutic composition for disrupting a biofilm, or treating an individual having a biofilm-associated disorder or disease, comprising a MMP.

16. The therapeutic composition of claim 15, wherein the MMP is selected from the group consisting of MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28, and combinations thereof.

17. The therapeutic composition of claim 15, wherein the MMP is a gelatinase.

18. The therapeutic composition of claim 15, wherein the therapeutic composition is formulated as a as a liquid, or as a paste, a slurry, a gel, a lotion, an emollient, a spray, a rinse, or an emulsion.

19. The therapeutic composition of claim 13, further comprising an antibiotic.

20. A kit comprising the therapeutic composition of claim 15, wherein the kit comprises instructions directing the use of the therapeutic composition for disrupting a biofilm and/or treating an individual for a biofilm-associated disease or disorder.
